(19) United States
(12) Patent Application Publication
(10) Pub. No.: US 2015/0057261 A1
(43) Pub. Date: Feb. 26, 2015

(54) AZETIDINE DERIVATIVES AS SPHINGOSINE-1 PHOSPHATE RECEPTORS MODULATORS

(71) Applicant: Allergan, Inc., Irvine, CA (US)

(72) Inventors: Wenkui K. Fang, Irvine, CA (US); Ken Chow, Newport Coast, CA (US); Evelyn G. Corpuz, Irvine, CA (US)

(73) Assignee: Allergan, Inc.

(21) Appl. No.: 14/463,755

(22) Filed: Aug. 20, 2014

Related U.S. Application Data
(60) Provisional application No. 61/868,233, filed on Aug. 21, 2013.

(51) Int. Cl. C07D 205/04

(52) U.S. Cl. CPC C07D 205/04
USPC 514/210.17; 548/953

(57) ABSTRACT

The present invention relates to azetidine derivatives, processes for preparing them, pharmaceutical compositions containing them and their use as pharmaceuticals as modulators of sphingosine-1-phosphate receptors.

### RELATED APPLICATION

This application claims the benefit of U.S. Provisional Patent Application Ser. No. 61/868,233 filed Aug. 21, 2013, the disclosure of which is hereby incorporated in its entirety by reference.

### FIELD OF THE INVENTION

The present invention relates to azetidine derivatives, processes for preparing them, pharmaceutical compositions containing them and their use as pharmaceuticals as modulators of sphingosine-1-phosphate receptors. The invention relates specifically to the use of these compounds and their pharmaceutical compositions to treat disorders associated with sphingosine-1-phosphate (S1P) receptor modulation.

### BACKGROUND OF THE INVENTION

Sphingosine-1 phosphate is stored in relatively high concentrations in human platelets, which lack the enzymes responsible for its catabolism, and it is released into the blood stream upon activation of physiological stimuli, such as growth factors, cytokines, and receptor agonists and antigens. It may also have a critical role in platelet aggregation and thrombosis and could aggravate cardiovascular diseases. On the other hand the relatively high concentration of the metabolite in high-density lipoproteins (HDL) may have beneficial implications for atherogenesis. For example, there are recent suggestions that sphingosine-1-phosphate, together with other lysolipids such as sphingosylphosphorylcholine and lysosulfatide, are responsible for the beneficial clinical effects of HDL by stimulating the production of the potent antiatherogenic signaling molecule nitric oxide by the vascular endothelium. In addition, like lysophosphatidic acid, it is a marker for certain types of cancer, and there is evidence that its role in cell division or proliferation may have an influence on the development of cancers. These are currently topics that are attracting great interest amongst medical researchers, and the potential for therapeutic intervention in sphingosine-1-phosphate metabolism is under active investigation.

### SUMMARY OF THE INVENTION

We have now discovered a group of novel compounds which are potent and selective sphingosine-1-phosphate modulators. As such, the compounds described herein are useful in treating a wide variety of disorders associated with modulation of sphingosine-1-phosphate receptors. The term “modulator” as used herein, includes but is not limited to: receptor agonist, antagonist, inverse agonist, inverse antagonist, partial agonist, partial antagonist.

This invention describes compounds of Formula I, which have sphingosine-1-phosphate receptor biological activity. The compounds in accordance with the present invention are thus of use in medicine, for example in the treatment of humans with diseases and conditions that are alleviated by S1P modulation.

In one aspect, the invention provides a compound having Formula I or a pharmaceutically acceptable salt thereof or stereoisomeric forms thereof, or the enantiomers, diastereoisomers, tautomers, zwitterions and pharmaceutically acceptable salts thereof:

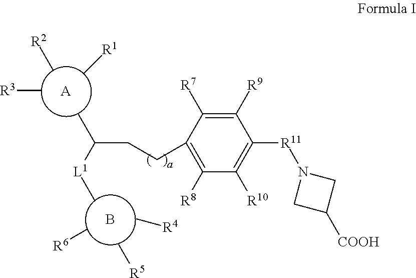

wherein:
A is substituted or unsubstituted C₆₋₁₀ aryl, substituted or unsubstituted heterocycle, substituted or unsubstituted C₃₋₈ cycloalkyl, or substituted or unsubstituted C₃₋₈ cycloalkenyl;
B is substituted or unsubstituted C₆₋₁₀ aryl, substituted or unsubstituted heterocycle, substituted or unsubstituted C₃₋₈ cycloalkyl, or substituted or unsubstituted C₃₋₈ cycloalkenyl;
R¹ is H, halogen, —OR¹², C₁₋₈ alkyl, CN, C(O)R¹³ or NR¹⁴R¹⁵;
R² is H, halogen, —OR¹², C₁₋₈ alkyl, CN, C(O)R¹³ or, NR¹⁴R¹⁵;
R³ is H, halogen, —OR¹², C₁₋₈ alkyl, CN, C(O)R¹³ or NR¹⁴R¹⁵;
R⁴ is H, halogen, —OR¹², C₁₋₈ alkyl, CN, C(O)R¹³ or NR¹⁴R¹⁵;
R⁵ is H, halogen, —OR¹², C₁₋₈ alkyl, CN, C(O)R¹³ or NR¹⁴R¹⁵;
R⁶ is H, halogen, —OR¹², C₁₋₈ alkyl, CN, C(O)R¹³ or NR¹⁴R¹⁵;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;

### R¹¹ is O, S or CH₂;

### L is CHR¹⁶, O, S or NR¹⁷;

a is 1, 2, 3 or 4;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl;
R¹⁶ is H, OH or C₁₋₈ alkyl; and
R¹⁷ is H or C₁₋₈ alkyl.

In another aspect, the invention provides a compound having Formula I wherein

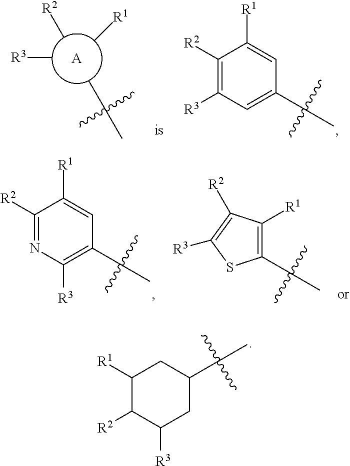

In another aspect, the invention provides a compound having Formula I wherein

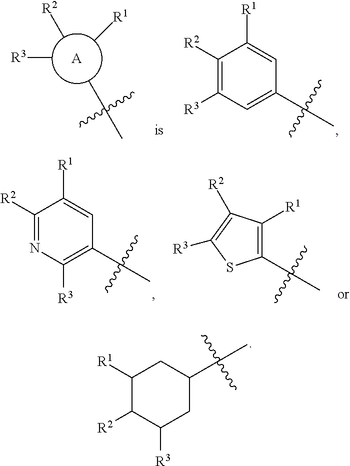

In another aspect, the invention provides a compound having Formula I wherein

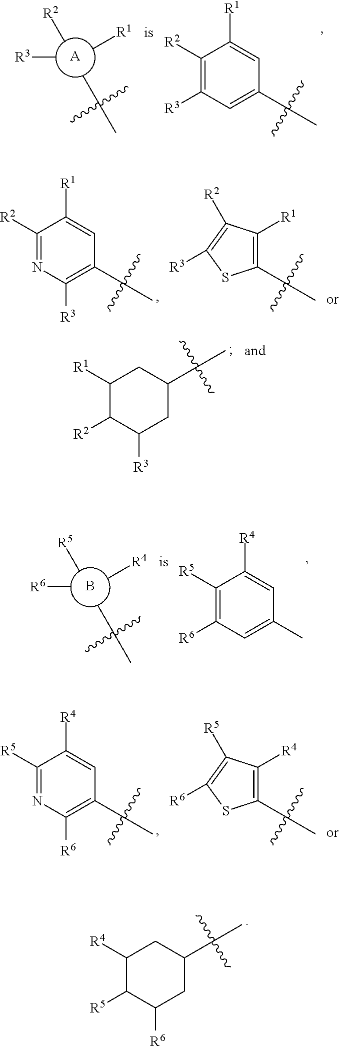

In another aspect, the invention provides a compound having Formula I wherein

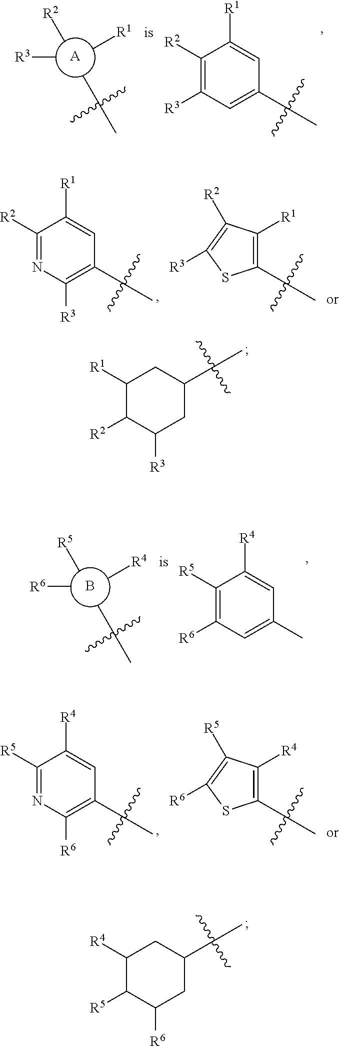

R¹ is H, halogen, —OR¹², C₁₋₈ alkyl, CN, C(O)R¹³ or NR¹⁴R¹⁵;
R² is H, halogen, —OR¹², C₁₋₈ alkyl, CN, C(O)R¹³ or, NR¹⁴R¹⁵;
R³ is H, halogen, —OR¹², C₁₋₈ alkyl, CN, C(O)R¹³ or NR¹⁴R¹⁵;
R⁴ is H, halogen, —OR¹², C₁₋₈ alkyl, CN, C(O)R¹³ or NR¹⁴R¹⁵;
R⁵ is H, halogen, —OR¹², C₁₋₈ alkyl, CN, C(O)R¹³ or NR¹⁴R¹⁵;
R⁶ is H, halogen, —OR¹², C₁₋₈ alkyl, CN, C(O)R¹³ or NR¹⁴R¹⁵;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;

### R¹¹ is O, S or CH₂;

### L is CHR¹⁶, O, S or NR¹⁷;

a is 1, 2, 3 or 4;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl;
R¹⁶ is H, OH or C₁₋₈ alkyl; and
R¹⁷ is H or C₁₋₈ alkyl.

In another aspect, the invention provides a compound having Formula I wherein

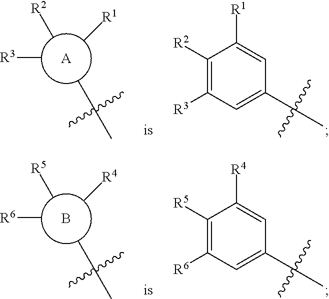

R¹ is H, halogen, —OR¹², C₁₋₈ alkyl, CN, C(O)R¹³ or NR¹⁴R¹⁵;
R² is H, halogen, —OR¹², C₁₋₈ alkyl, CN, C(O)R¹³ or, NR¹⁴R¹⁵;
R³ is H, halogen, —OR¹², C₁₋₈ alkyl, CN, C(O)R¹³ or NR¹⁴R¹⁵;
R⁴ is H, halogen, —OR¹², C₁₋₈ alkyl, CN, C(O)R¹³ or NR¹⁴R¹⁵;
R⁵ is H, halogen, —OR¹², C₁₋₈ alkyl, CN, C(O)R¹³ or NR¹⁴R¹⁵;
R⁶ is H, halogen, —OR¹², C₁₋₈ alkyl, CN, C(O)R¹³ or NR¹⁴R¹⁵;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;

### R¹¹ is O, S or CH₂;

### L is CHR¹⁶, O, S or NR¹⁷;

a is 1, 2, 3 or 4;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl;
R¹⁶ is H, OH or C₁₋₈ alkyl; and
R¹⁷ is H or C₁₋₈ alkyl.

In another aspect, the invention provides a compound having Formula I wherein

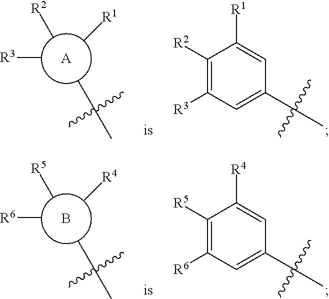

R¹ is H, halogen;
R² is H, halogen;
R³ is H, halogen;
R⁴ is H, C₁₋₈ alkyl;
R⁵ is H, C₁₋₈ alkyl;
R⁶ is H, C₁₋₈ alkyl;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;

### R¹¹ is CH₂;

### L is CHR¹⁶;

a is 1, 2;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl; and
R¹⁶ is H, OH or C₁₋₈ alkyl.

In another aspect, the invention provides a compound having Formula I wherein

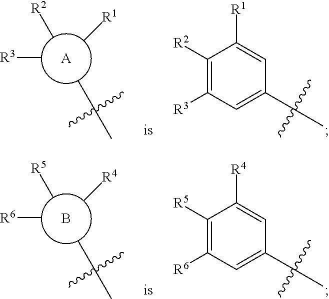

R¹ is H, halogen;
R² is H, halogen;
R³ is H, halogen;
R⁴ is H, C₁₋₈ alkyl;
R⁵ is H, C₁₋₈ alkyl;
R⁶ is H, C₁₋₈ alkyl;
R⁷ is H, halogen, C₁₋₈ alkyl;
R⁸ is H, halogen, C₁₋₈ alkyl;
R⁹ is H, halogen, —C₁₋₈ alkyl;
R¹⁰ is H, halogen, —C₁₋₈ alkyl;

R¹¹ is CH₂;

### L is CHR¹⁶;

a is 1 or 2; and

### R¹⁶ is H.

In another aspect, the invention provides a compound having Formula I wherein

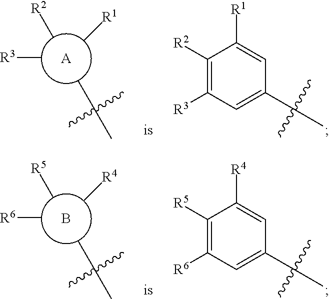

R¹ is halogen;

### R² is H;

R³ is H, halogen;
R⁴ is C₁₋₈ alkyl;
R⁵ is C₁₋₈ alkyl;

### R⁶ is H;

### R⁷ is H;

### R⁸ is H;

### R⁹ is H;

### R¹⁰ is H;

### R¹¹ is CH₂;

### L is CHR¹⁶;

a is 1 or 2; and

### R¹⁶ is H.

In another aspect, the invention provides a compound having Formula I wherein

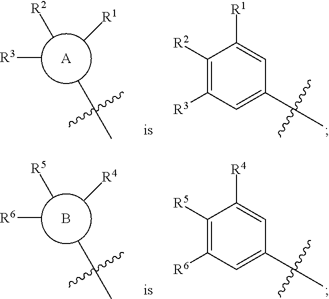

### R¹ is Cl or F;

### R² is H;

### R³ is H or F;

R⁴ is methyl;
R⁵ is methyl;

### R⁶ is H;

### R⁷ is H;

### R⁸ is H;

### R⁹ is H;

### R¹⁰ is H;

### R¹¹ is CH₂;

### L is CHR¹⁶;

a is 1 or 2; and

### R¹⁶ is H.

The term “alkyl”, as used herein, refers to saturated, monovalent or divalent hydrocarbon moieties having linear or branched moieties or combinations thereof and containing 1 to 8 carbon atoms. One methylene (—CH₂—) group, of the alkyl group can be replaced by oxygen, sulfur, sulfoxide, nitrogen, carbonyl, carboxyl, sulfonyl, sulfate, sulfonate, amide, sulfonamide, by a divalent C₃₋₈ cycloalkyl, by a divalent heterocycle, or by a divalent aryl group. Alkyl groups can have one or more chiral centers. Alkyl groups can be independently substituted by halogen atoms, hydroxyl groups, cycloalkyl groups, amino groups, heterocyclic groups, aryl groups, carboxylic acid groups, phosphonic acid groups, sulphonic acid groups, phosphoric acid groups, nitro groups, amide groups, sulfonamide groups.

The term “cycloalkyl”, as used herein, refers to a monovalent or divalent group of 3 to 8 carbon atoms derived from a saturated cyclic hydrocarbon. Cycloalkyl groups can be monocyclic or polycyclic. Cycloalkyl can be independently substituted by halogen atoms, sulfonyl C₁₋₈ alkyl groups, sulfoxide C₁₋₈ alkyl groups, sulfonamide groups, nitro groups, cyano groups, —OC₁₋₈ alkyl groups, —SC₁₋₈ alkyl groups, —C₁₋₈ alkyl groups, —C₂₋₆ alkenyl groups, —C₂₋₆ alkynyl groups, ketone groups, alkylamino groups, amino groups, aryl groups, C₃₋₈ cycloalkyl groups or hydroxyl groups.

The term “cycloalkenyl”, as used herein, refers to a monovalent or divalent group of 3 to 8 carbon atoms derived from a saturated cycloalkyl having at least one double bond. Cycloalkenyl groups can be monocyclic or polycyclic. Cycloalkenyl groups can be independently substituted by halogen atoms, sulfonyl groups, sulfoxide groups, nitro groups, cyano groups, —OC₁₋₆ alkyl groups, —SC₁₋₆ alkyl groups, —C₁₋₆ alkyl groups, —C₂₋₆ alkenyl groups, —C₂₋₆ alkynyl groups, ketone groups, alkylamino groups, amino groups, aryl groups, C₃₋₈ cycloalkyl groups or hydroxyl groups.

The term “halogen”, as used herein, refers to an atom of chlorine, bromine, fluorine, iodine.

The term “alkenyl”, as used herein, refers to a monovalent or divalent hydrocarbon radical having 2 to 6 carbon atoms, derived from a saturated alkyl, having at least one double bond. One methylene (—CH₂—) group, of the alkenyl can be replaced by oxygen, sulfur, sulfoxide, nitrogen, carbonyl, carboxyl, sulfonyl, sulfate, sulfonate, amide, sulfonamide, by a divalent C₃₋₈ cycloalkyl, by a divalent heterocycle, or by a divalent aryl group. C₂₋₆ alkenyl can be in the E or Z configuration. Alkenyl groups can be substituted by alkyl groups, as defined above or by halogen atoms.

The term “alkynyl”, as used herein, refers to a monovalent or divalent hydrocarbon radical having 2 to 6 carbon atoms, derived from a saturated alkyl, having at least one triple bond. One methylene (—CH₂—) group, of the alkynyl can be replaced by oxygen, sulfur, sulfoxide, nitrogen, carbonyl, carboxyl, sulfonyl, sulfate, sulfonate, amide, sulfonamide, by a divalent C₃₋₈ cycloalkyl, by a divalent heterocycle, or by a divalent aryl group. Alkynyl groups can be substituted by alkyl groups, as defined above, or by halogen atoms.

The term “heterocycle” as used herein, refers to a 3 to 10 membered ring, which can be aromatic or non-aromatic, saturated or unsaturated, containing at least one heteroatom selected form oxygen, nitrogen, sulfur, or combinations of at least two thereof, interrupting the carbocyclic ring structure. The heterocyclic ring can be interrupted by a C═O; the S and N heteroatoms can be oxidized. Heterocycles can be monocyclic or polycyclic. Heterocyclic ring moieties can be substituted by halogen atoms, sulfonyl groups, sulfoxide groups, nitro groups, cyano groups, —OC₁₋₆ alkyl groups, —SC₁₋₆ alkyl groups, —C₁₋₈ alkyl groups, —C₂₋₆ alkenyl groups, —C₂₋₆ alkynyl groups, ketone groups, alkylamino groups, amino groups, aryl groups, C₃₋₈ cycloalkyl groups or hydroxyl groups.

The term “aryl” as used herein, refers to an organic moiety derived from an aromatic hydrocarbon consisting of a ring containing 6 to 10 carbon atoms, by removal of one hydrogen atom. Aryl can be substituted by halogen atoms, sulfonyl C₁₋₆ alkyl groups, sulfoxide C₁₋₆ alkyl groups, sulfonamide groups, carboxcyclic acid groups, C₁₋₆ alkyl carboxylates (ester) groups, amide groups, nitro groups, cyano groups, —OC₁₋₆ alkyl groups, —SC₁₋₆ alkyl groups, —C₁₋₆ alkyl groups, —C₂₋₆ alkenyl groups, —C₂₋₆ alkynyl groups, ketone groups, aldehydes, alkylamino groups, amino groups, aryl groups, C₃₋₈ cycloalkyl groups or hydroxyl groups. Aryls can be monocyclic or polycyclic.

The term “hydroxyl” as used herein, represents a group of formula “—OH”.

The term “carbonyl” as used herein, represents a group of formula “—C(O)—”.

The term “ketone” as used herein, represents an organic compound having a carbonyl group linked to a carbon atom such as —(CO)R^{x} wherein R^{x} can be alkyl, aryl, cycloalkyl, cycloalkenyl, heterocycle as defined above.

The term “amine” as used herein, represents a group of formula “—NR^{x}R^{y}”, wherein R^{x} and R^{y} can be the same or independently H, alkyl, aryl, cycloalkyl, cycloalkenyl, heterocycle as defined above.

The term “carboxyl” as used herein, represents a group of formula “—C(O)O—”.

The term “sulfonyl” as used herein, represents a group of formula “—SO₂⁻”.

The term “sulfate” as used herein, represents a group of formula “—O—S(O)₂—O—”.

The term “sulfonate” as used herein, represents a group of the formula “—S(O)₂—O—”.

The term “carboxylic acid” as used herein, represents a group of formula “—C(O)OH”.

The term “nitro” as used herein, represents a group of formula “—NO₂”.

The term “cyano” as used herein, represents a group of formula “—CN”.

The term “amide” as used herein, represents a group of formula “—C(O)NR^{x}R^{y},” wherein R^{x} and R^{y} can be the same or independently H, alkyl, aryl, cycloalkyl, cycloalkenyl, heterocycle as defined above.

The term “sulfonamide” as used herein, represents a group of formula “—S(O)₂NR^{x}R^{y}” wherein R^{x} and R^{y} can be the same or independently H, alkyl, aryl, cycloalkyl, cycloalkenyl, heterocycle as defined above.

The term “sulfoxide” as used herein, represents a group of formula “—S(O)—”.

The term “phosphonic acid” as used herein, represents a group of formula “—P(O)(OH)₂”.

The term “phosphoric acid” as used herein, represents a group of formula “—OP(O)(OH)₂”.

The term “sulphonic acid” as used herein, represents a group of formula “—S(O)₂OH”.

The formula “H”, as used herein, represents a hydrogen atom.

The formula “O”, as used herein, represents an oxygen atom.

The formula “N”, as used herein, represents a nitrogen atom.

The formula “S”, as used herein, represents a sulfur atom.

Compounds of the invention are:
1-{4-[4-(3-chlorophenyl)-5-(3,4-dimethylphenyl)pentyl]benzyl}azetidine-3-carboxylic acid;
1-{4-[5-(3,5-difluorophenyl)-6-(3,4-dimethylphenyl)hexyl]benzyl}azetidine-3-carboxylic acid; and
1-{4-[5-(3-chlorophenyl)-6-(3,4-dimethylphenyl)hexyl]benzyl}azetidine-3-carboxylic acid.

Some compounds of Formula I and some of their intermediates have at least one stereogenic center in their structure. This stereogenic center may be present in an R or S configuration, said R and S notation is used in correspondence with the rules described in Pure Appli. Chem. (1976), 45, 11-13.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of Formula I are able to form.

The acid addition salt form of a compound of Formula I that occurs in its free form as a base can be obtained by treating the free base with an appropriate acid such as an inorganic acid, such as for example, hydrochloric acid, hydrobromic acid, sulfuric acid, phosphoric acid, nitric acid and the like; or an organic acid such as for example, acetic, hydroxyacetic, propanoic, lactic, pyruvic, malonic, fumaric acid, maleic acid, oxalic acid, tartaric acid, succinic acid, malic acid, ascorbic acid, benzoic acid, tannic acid, pamoic acid, citric, methylsulfonic, ethanesulfonic, benzenesulfonic, formic and the like (Handbook of Pharmaceutical Salts, P. Heinrich Stahl & Camille G. Wermuth (Eds), Verlag Helvetica Chimica Acta-Zürich, 2002, 329-345).

The base addition salt form of a compound of Formula I that occurs in its acid form can be obtained by treating the acid with an appropriate base such as an inorganic base, for example, sodium hydroxide, magnesium hydroxide, potassium hydroxide, calcium hydroxide, ammonia and the like; or an organic base such as for example, L-Arginine, ethanolamine, betaine, benzathine, morpholine and the like. (Handbook of Pharmaceutical Salts, P. Heinrich Stahl & Camille G. Wermuth (Eds), Verlag Helvetica Chimica Acta-Zürich, 2002, 329-345).

Compounds of Formula I and their salts can be in the form of a solvate, which is included within the scope of the present invention. Such solvates include for example hydrates, alcoholates and the like.

The compounds of the invention are indicated for use in treating or preventing conditions in which there is likely to be a component involving the sphingosine-1-phosphate receptors.

In another embodiment, there are provided pharmaceutical compositions including at least one compound of the invention in a pharmaceutically acceptable carrier.

In a further embodiment of the invention, there are provided methods for treating disorders associated with modulation of sphingosine-1-phosphate receptors. Such methods can be performed, for example, by administering to a subject in need thereof a pharmaceutical composition containing a therapeutically effective amount of at least one compound of the invention.

These compounds are useful for the treatment of mammals, including humans, with a range of conditions and diseases that are alleviated by S1P modulation: not limited to the treatment of diabetic retinopathy, other retinal degenerative conditions, dry eye, angiogenesis and wounds.

Therapeutic utilities of S1P modulators are ocular diseases, such as but not limited to: wet and dry age-related macular degeneration, diabetic retinopathy, angiogenesis inhibition, retinopathy of prematurity, retinal edema, geographic atrophy, glaucomatous optic neuropathy, chorioretinopathy, hypertensive retinopathy, ocular ischemic syndrome, prevention of inflammation-induced fibrosis in the back of the eye, various ocular inflammatory diseases including uveitis, scleritis, keratitis, and retinal vasculitis; or systemic vascular barrier related diseases such as but not limited to: various inflammatory diseases, including acute lung injury, its prevention, sepsis, tumor metastasis, atherosclerosis, pulmonary edemas, and ventilation-induced lung injury; or autoimmune diseases and immunosuppression such as but not limited to: rheumatoid arthritis, Crohn's disease, Graves' disease, inflammatory bowel disease, multiple sclerosis, Myasthenia gravis, Psoriasis, ulcerative colitis, autoimmune uveitis, renal ischemia/perfusion injury, contact hypersensitivity, atopic dermatitis, and organ transplantation; or allergies and other inflammatory diseases such as but not limited to: urticaria, bronchial asthma, and other airway inflammations including pulmonary emphysema and chronic obstructive pulmonary diseases; or cardiac protection such as but not limited to: ischemia reperfusion injury and atherosclerosis; or wound healing such as but not limited to: scar-free healing of wounds from cosmetic skin surgery, ocular surgery, GI surgery, general surgery, oral injuries, various mechanical, heat and burn injuries, prevention and treatment of photoaging and skin ageing, and prevention of radiation-induced injuries; or bone formation such as but not limited to: treatment of osteoporosis and various bone fractures including hip and ankles; or anti-nociceptive activity such as but not limited to: visceral pain, pain associated with diabetic neuropathy, rheumatoid arthritis, chronic knee and joint pain, tendonitis, osteoarthritis, neuropathic pains; or central nervous system neuronal activity in Alzheimer's disease, age-related neuronal injuries; or in organ transplant such as renal, corneal, cardiac or adipose tissue transplant.

In still another embodiment of the invention, there are provided methods for treating disorders associated with modulation of sphingosine-1-phosphate receptors. Such methods can be performed, for example, by administering to a subject in need thereof a therapeutically effective amount of at least one compound of the invention, or any combination thereof, or pharmaceutically acceptable salts, hydrates, solvates, crystal forms and individual isomers, enantiomers, and diastereoisomers thereof.

The present invention concerns the use of a compound of Formula I or a pharmaceutically acceptable salt thereof, for the manufacture of a medicament for the treatment of ocular disease, wet and dry age-related macular degeneration, diabetic retinopathy, retinopathy of prematurity, retinal edema, geographic atrophy, angiogenesis inhibition, glaucomatous optic neuropathy, chorioretinopathy, hypertensive retinopathy, ocular ischemic syndrome, prevention of inflammation-induced fibrosis in the back of the eye, various ocular inflammatory diseases including uveitis, scleritis, keratitis, and retinal vasculitis; or systemic vascular barrier related diseases, various inflammatory diseases, including acute lung injury, its prevention, sepsis, tumor metastasis, atherosclerosis, pulmonary edemas, and ventilation-induced lung injury; or autoimmune diseases and immunosuppression, rheumatoid arthritis, Crohn's disease, Graves' disease, inflammatory bowel disease, multiple sclerosis, Myasthenia gravis, Psoriasis, ulcerative colitis, autoimmune uveitis, renal ischemia/perfusion injury, contact hypersensitivity, atopic dermatitis, and organ transplantation; or allergies and other inflammatory diseases, urticaria, bronchial asthma, and other airway inflammations including pulmonary emphysema and chronic obstructive pulmonary diseases; or cardiac protection, ischemia reperfusion injury and atherosclerosis; or wound healing, scar-free healing of wounds from cosmetic skin surgery, ocular surgery, GI surgery, general surgery, oral injuries, various mechanical, heat and burn injuries, prevention and treatment of photoaging and skin ageing, and prevention of radiation-induced injuries; or bone formation, treatment of osteoporosis and various bone fractures including hip and ankles; or anti-nociceptive activity, visceral pain, pain associated with diabetic neuropathy, rheumatoid arthritis, chronic knee and joint pain, tendonitis, osteoarthritis, neuropathic pains; or central nervous system neuronal activity in Alzheimer's disease, age-related neuronal injuries; or in organ transplant such as renal, corneal, cardiac or adipose tissue transplant.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

Pharmaceutical compositions containing invention compounds may be in a form suitable for topical use, for example, as oily suspensions, as solutions or suspensions in aqueous liquids or nonaqueous liquids, or as oil-in-water or water-in-oil liquid emulsions. Pharmaceutical compositions may be prepared by combining a therapeutically effective amount of at least one compound according to the present invention, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.0001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants. Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner an ophthalmically acceptable antioxidant for use in the present invention includes, but is not limited to, sodium metabisulfite, sodium thiosulfate, acetylcysteine, butylated hydroxyanisole and butylated hydroxytoluene. Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 0-10 |
| buffer | 0.01-10 |
| pH adjustor | q.s. pH 4.5-7.8 |
| antioxidant | as needed |
| surfactant | as needed |
| purified water | to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for drop wise application are usually made of suitable inert, non-toxic plastic material, and generally contain between about 0.5 and about 15 ml solution. One package may contain one or more unit doses. Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl.

Invention compounds may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

The compounds and pharmaceutical compositions described herein are useful as medicaments in mammals, including humans, for treatment of diseases and/or alleviations of conditions which are responsive to treatment by agonists or functional antagonists of sphingosine-1-phosphate receptors. Thus, in further embodiments of the invention, there are provided methods for treating a disorder associated with modulation of sphingosine-1-phosphate receptors. Such methods can be performed, for example, by administering to a subject in need thereof a pharmaceutical composition containing a therapeutically effective amount of at least one invention compound. As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

The present invention concerns also processes for preparing the compounds of Formula I. The compounds of Formula I according to the invention can be prepared analogously to conventional methods as understood by the person skilled in the art of synthetic organic chemistry. The synthetic schemes set forth below, illustrate how compounds according to the invention can be made.

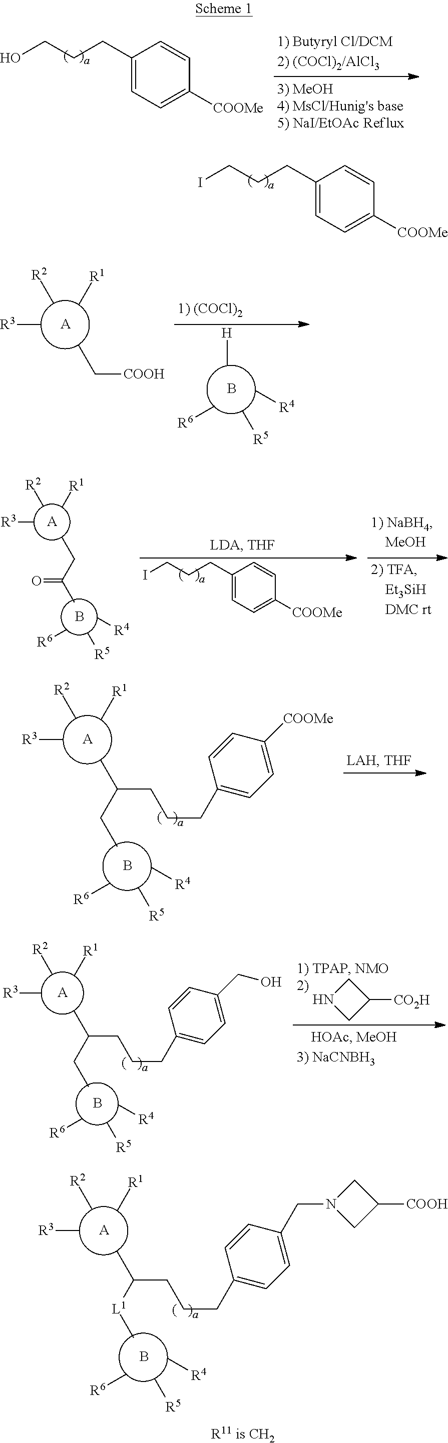

A suitably substituted acid chloride and a suitably substituted aryl were mixed and cooled to −78° C., AlCl₃ (1.5 eq) was added, the resulting reaction mixture was stirred at this temperature for 60 minutes, then quenched with concentrated HCl. The reaction mixture thus created was diluted with ethyl acetate and washed with brine, then concentrated. Flash chromatography gave the corresponding ketone. The ketone was added to LDA (prepared fresh from n-BuLi and diisopropylamine in THF) at −78° C., then warmed to 0° C. and stirred for 60 minutes. The reaction mixture was cooled to −78° C. again, a suitably substituted iodoalkylbenzoic acid ester was added, the resultant mixture was allowed to warm to room temperature and stirred for 4 hours, then heated to 95° C. and stirred for another 14 hours. The iodoalkylbenzoic acid ester was obtained according to the procedure described in J. Med. Chem. 2008, 51, 2954-290. After being diluted with ethyl acetate, the resulting reaction mixture was washed with saturated ammonium chloride and brine, then dried (NaSO₄) and concentrated. This alkylated ketone was then dissolved in methanol and cooled to 0° C., sodium borohydride (1.0 eq) was slowly added. The reaction mixture was stirred at 0° C. for 10 minutes, then diluted with ethyl acetate and the resultant organic phase was washed with saturated ammonium chloride and brine, then dried and concentrated. Flash chromatography gave the desired compound which was dissolved in dichloromethane and cooled to 0° C. Trifluoroacetic acid (2 eq) was added, followed by the addition of triethylsilane. The resulting reaction mixture was allowed to warm to room temperature and stirred for 4 hours, then concentrated. This intermediate ester was dissolved in ether, then cooled to 0° C. LAH (1.5 eq) was added, the resulting reaction mixture was stirred at the same temperature for 60 minutes, then diluted HCl was added. The organic phase was separated and washed with brine, then dried (NaSO₄) and concentrated. Flash chromatography gave the corresponding alcohol, which was mixed with NMO (26 eq), molecular sieves (500 mg) in AcCN:DCM (1:5). A catalytic amount of TPAP (35 mg) was added. The resulting reaction mixture was stirred at RT for 1 hour and then evaporated to dryness. The desired aldehyde compound was purified by MPLC using 0-10% ethyl acetate in hexane and used immediately in the subsequent transformation.

The aldehyde (1 eq), azetidine acid (1.3 eq) and TEA (1.3 eq) were mixed with MeOH (10 ml). Upon stirring at 60° C. for 90 min, the reaction solution was cooled to RT. NaBH₃CN was added and stirred at RT for 2 hour. The reaction mixture was quenched with 0.5 mL of water and concentrated to minimal amount. The corresponding compound was isolated by reverse phase MPLC using 0 to 90% H₂O in AcCN.

The following abbreviations are used in the general schemes and in the examples:

AlCl₃ aluminum chloride
HCl hydrochloric acid
NaH sodium hydrate
NaSO₄ sodium sulfate
LDA lithium diisopropylamide
n-BuLi n-butyllithium
THF tetrahydrofuran

### NMO N-methylmorpholine-N-Oxide

LAH lithium aluminum hydride
AcCN acetonitrile
DCM dichloromethane
TPAP tetrapropylammonium perruthenate
MPLC medium pressure liquid chromatography
TEA triethylamine
MeOH methanol
NaBH₄ sodium borohydrate
RT room temperature
NaCNBH₃ sodium cyanoborohydride
CDCl₃ deuterated chloroform
CD₃OD deuterated methanol
DMF dimethylformamide
HOAc acetic acid
NaI sodium iodide
EtOAc ethyl acetate
MsCl methanesulfonyl chloride
(COCl)₂ oxalylchloride
Hunig's base N,N-diisopropylethylamine

Those skilled in the art will be able to routinely modify and/or adapt the Scheme 1 to synthesize any compounds of the invention covered by Formula I.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise.

It will be readily apparent to those skilled in the art that some of the compounds of the invention may contain one or more asymmetric centers, such that the compounds may exist in enantiomeric as well as in diastereoisomeric forms. Unless it is specifically noted otherwise, the scope of the present invention includes all enantiomers, diastereoisomers and racemic mixtures. Some of the compounds of the invention may form salts with pharmaceutically acceptable acids or bases, and such pharmaceutically acceptable salts of the compounds described herein are also within the scope of the invention.

The present invention includes all pharmaceutically acceptable isotopically enriched compounds. Any compound of the invention may contain one or more isotopic atoms enriched or different than the natural ratio such as deuterium ²H (or D) in place of protium ¹H (or H) or use of ¹³C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

The following examples are for illustrative purposes only and are not intended, nor should they be construed as limiting the invention in any manner. Those skilled in the art will appreciate that variations and modifications of the following examples can be made without exceeding the spirit or scope of the invention.

As will be evident to those skilled in the art, individual isomeric forms can be obtained by separation of mixtures thereof in conventional manner. For example, in the case of diasteroisomeric isomers, chromatographic separation may be employed.

Compound names were generated with ACD product version 12.5; and Intermediates and reagent names used in the examples were generated with software such as Chem Bio Draw Ultra version 12.0 or Auto Nom 2000 from MDL ISIS Draw 2.5 SP1. In general, characterization of the compounds is performed according to the following methods: NMR spectra are recorded on 300 and/or 600 MHz Varian and acquired at room temperature. Chemical shifts are given in ppm referenced either to internal TMS or to the solvent signal.

All the reagents, solvents, catalysts for which the synthesis is not described are purchased from chemical vendors such as Sigma Aldrich, Fluka, Bio-Blocks, Combi-blocks, TCI, VWR, Lancaster, Oakwood, Trans World Chemical, Alfa, Fisher, AK Scientific, AmFine Com, Carbocore, Maybridge, Frontier, Matrix, Ukrorgsynth, Toronto, Ryan Scientific, SiliCycle, Anaspec, Syn Chem, Chem-Impex, MIC-scientific, Ltd; however some known intermediates, were prepared according to published procedures.

The subsequent examples were prepared following the general procedure described above. Usually the compounds of the invention were purified by column chromatography (Auto-column) on an Teledyne-ISCO CombiFlash with a silica column, unless noted otherwise. Some compounds of this invention can generally be prepared in one step from commercially available literature starting materials.

### Example 1

### Intermediate 1

### 4-[5-(3,5-Difluorophenyl)-6-(3,4-dimethylphenyl)hexyl]benzaldehyde

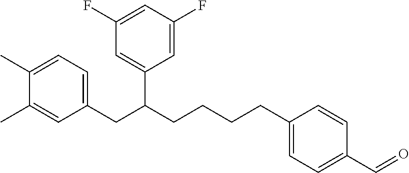 [SMILES: Cc1ccc(CC(CCCCc2ccc(C=O)cc2)c2cc(F)cc(F)c2)cc1C]

¹H NMR (300 MHz, CDCl₃) δ ppm 1.00-1.33 (m, 2H) 1.39-1.83 (m, 4H) 2.18 (s, 6H) 2.48-2.66 (m, 2H) 2.74 (br. s, 3H) 6.62 (d, J=7.62 Hz, 3H) 6.73 (d, J=7.62 Hz, 1H) 6.78 (br. s, 1H) 6.97 (d, J=7.62 Hz, 1H) 7.24 (d, J=8.20 Hz, 2H) 7.76 (d, J=7.91 Hz, 2H) 9.95 (s, 1H).

### Example 2

### Intermediate 2

### {4-[4-(3-Chlorophenyl)-5-(3,4-dimethylphenyl)pentyl]phenyl}methanol

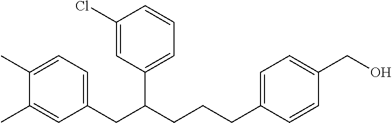 [SMILES: Cc1ccc(CC(CCCc2ccc(CO)cc2)c2cccc(Cl)c2)cc1C]

¹H NMR (300 MHz, CDCl₃) δ ppm 1.35-1.50 (m, 2H) 1.51-1.80 (m, 2H) 2.19 (d, J=3.52 Hz, 6H) 2.40-2.64 (m, 2H) 2.69-2.84 (m, 3H) 4.63 (s, 2H) 6.73 (d, J=7.62 Hz, 1H) 6.78 (br. s, 1H) 6.96 (d, J=7.03 Hz, 2H) 6.99-7.10 (m, 3H) 7.11-7.19 (m, 2H) 7.23 (d, J=7.91 Hz, 2H).

### Example 3

### Intermediate 3

### 4-[4-(3-Chlorophenyl)-5-(3,4-dimethylphenyl)pentyl]benzaldehyde

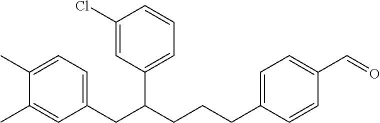 [SMILES: Cc1ccc(CC(CCCc2ccc(C=O)cc2)c2cccc(Cl)c2)cc1C]

¹H NMR (300 MHz, CDCl₃) δ ppm 1.34-1.53 (m, 2H) 1.52-1.83 (m, 2H) 2.18 (br. s., 3H) 2.20 (br. s., 3H) 2.43-2.68 (m, 2H) 2.68-2.85 (m, 3H) 6.73 (d, J=7.62 Hz, 1H) 6.78 (br. s, 1H) 6.96 (d, J=7.33 Hz, 2H) 7.09 (br. s, 1H) 7.12-7.23 (m, 4H) 7.73 (d, J=7.91 Hz, 2H) 9.94 (s, 1H).

### Example 4

### Intermediate 4

### {4-[5-(3-Chlorophenyl)-6-(3,4-dimethylphenyl)hexyl]phenyl}methanol

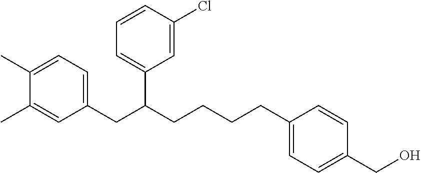 [SMILES: Cc1ccc(CC(CCCCc2ccc(CO)cc2)c2cccc(Cl)c2)cc1C]

¹H NMR (300 MHz, CDCl₃) δ ppm 1.06-1.25 (m, 2H) 1.43-1.74 (m, 4H) 2.19 (br. s, 6H) 2.39-2.58 (m, 2H) 2.67-2.83 (m, 3H) 4.63 (br. s, 2H) 6.74 (d, J=7.91 Hz, 1H) 6.79 (s, 1H) 6.96 (d, J=7.62 Hz, 2H) 7.04-7.19 (m, 5H) 7.23 (d, J=7.91 Hz, 2H).

### Example 5

### Intermediate 5

### 4-[5-(3-Chlorophenyl)-6-(3,4-dimethylphenyl)hexyl]benzaldehyde

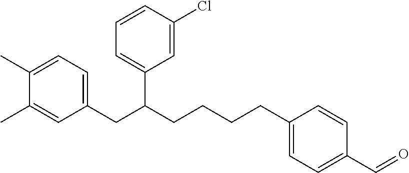 [SMILES: Cc1ccc(CC(CCCCc2ccc(C=O)cc2)c2cccc(Cl)c2)cc1C]

¹H NMR (300 MHz, CDCl₃) δ ppm 1.07-1.27 (m, 2H) 1.44-1.65 (m, 3H) 1.65-1.77 (m, 1H) 2.21 (d, J=5.28 Hz, 6H) 2.51-2.65 (m, 2H) 2.71-2.85 (m, 3H) 6.76 (d, J=7.63 Hz, 1H) 6.81 (br. s, 1H) 6.95-7.03 (m, 2H) 7.09-7.14 (m, 1H) 7.15-7.21 (m, 2H) 7.24 (d, J=8.07 Hz, 2H) 7.77 (d, J=8.22 Hz, 2H) 9.97 (s, 1H).

### Example 6

### Compound 1

### 1-{4-[5-(3-Chlorophenyl)-6-(3,4-dimethylphenyl)hexyl]benzyl}azetidine-3-carboxylic acid

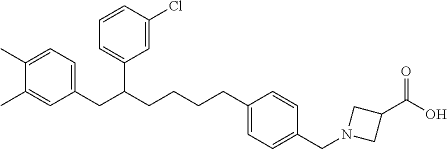 [SMILES: Cc1ccc(CC(CCCCc2ccc(CN3CC(C(=O)O)C3)cc2)c2cccc(Cl)c2)cc1C]

¹H NMR (300 MHz, CDCl₃) δ ppm 1.03-1.22 (m, 2H) 1.35-1.76 (m, 4H) 2.18 (s, 6H) 2.35-2.53 (m, 2H) 2.61-2.83 (m, 3H) 3.20-3.46 (m, 1H) 3.85-4.02 (m, 2H) 4.08 (br. s., 4H) 6.73 (d, J=7.91 Hz, 1H) 6.78 (br. s, 1H) 6.95 (d, J=7.62 Hz, 2H) 7.02-7.18 (m, 5H) 7.27 (d, J=7.62 Hz, 2H).

### Example 7

### Compound 2

### 1-{4-[4-(3-Chlorophenyl)-5-(3,4-dimethylphenyl)pentyl]benzyl}azetidine-3-carboxylic acid

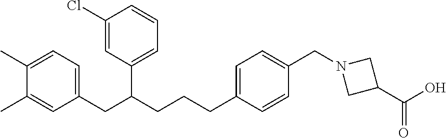 [SMILES: Cc1ccc(CC(CCCc2ccc(CN3CC(C(=O)O)C3)cc2)c2cccc(Cl)c2)cc1C]

¹H NMR (300 MHz, CD₃OD) δ ppm 1.29-1.47 (m, 2H) 1.50-1.75 (m, 2H) 2.13 (s, 6H) 2.35-2.59 (m, 2H) 2.63-2.87 (m, 3H) 3.23-3.46 (m, 1H) 4.12 (d, J=8.20 Hz, 4H) 4.24 (s, 2H) 6.69 (d, J=7.62 Hz, 1H) 6.74 (br. s, 1H) 6.89 (d, J=7.62 Hz, 1H) 6.97 (d, J=7.33 Hz, 1H) 7.03-7.17 (m, 5H) 7.28 (d, J=7.62 Hz, 2H).

### Example 8

### Compound 3

### 1-{4-[5-(3,5-Difluorophenyl)-6-(3,4-dimethylphenyl)hexyl]benzyl}azetidine-3-carboxylic acid

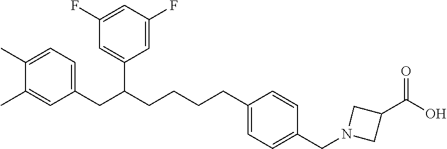 [SMILES: Cc1ccc(CC(CCCCc2ccc(CN3CC(C(=O)O)C3)cc2)c2cc(F)cc(F)c2)cc1C]

¹H NMR (300 MHz, CDCl₃) δ ppm 1.04-1.25 (m, 2H) 1.37-1.79 (m, 4H) 2.18 (br. s, 6H) 2.35-2.57 (m, 2H) 2.74 (br. s, 3H) 3.20-3.44 (m, 1H) 3.86-4.04 (m, 2H) 4.09 (br. s., 4H) 6.50-6.66 (m, 3H) 6.72 (d, J=7.62 Hz, 1H) 6.77 (br. s, 1H) 6.95 (d, J=7.62 Hz, 1H) 7.07 (d, J=7.91 Hz, 2H) 7.28 (d, J=8.20 Hz, 2H).

### Example 9

### Biological Data

Compounds were synthesized and tested for S1P1 activity using the GTP γ³⁵S binding assay. These compounds may be assessed for their ability to activate or block activation of the human S1P1 receptor in cells stably expressing the S1P1 receptor.

GTP γ³⁵S binding was measured in the medium containing (mM) HEPES 25, pH 7.4, MgCl₂ 10, NaCl 100, dithiothreitol 0.5, digitonin 0.003%, 0.2 nM GTP γ³⁵S, and 5 μg membrane protein in a volume of 150 μl. Test compounds were included in the concentration range from 0.08 to 5,000 nM unless indicated otherwise. Membranes were incubated with 100 μM 5′-adenylylimidodiphosphate for 30 min, and subsequently with 10 μM GDP for 10 min on ice. Drug solutions and membrane were mixed, and then reactions were initiated by adding GTP γ³⁵S and continued for 30 min at 25° C. Reaction mixtures were filtered over Whatman GF/B filters under vacuum, and washed three times with 3 mL of ice-cold buffer (HEPES 25, pH7.4, MgCl₂ 10 and NaCl 100). Filters were dried and mixed with scintillant, and counted for ³⁵S activity using a β-counter. Agonist-induced GTP γ³⁵S binding was obtained by subtracting that in the absence of agonist. Binding data were analyzed using a non-linear regression method. In case of antagonist assay, the reaction mixture contained 10 nM S1P1 in the presence of test antagonist at concentrations ranging from 0.08 to 5000 nM.

Table 1 shows activity potency: S1P1 receptor from GTP γ³⁵S: nM, (EC₅₀)

TABLE 1

| | S1P1 |
|---|---|
| | EC₅₀ |
| IUPAC name | (nM) |
| 1-{4-[4-(3-chlorophenyl)-5-(3,4- | 17.15 |
| dimethylphenyl)pentyl]benzyl}azetidine-3-carboxylic acid | |
| 1-{4-[5-(3,5-difluorophenyl)-6-(3,4- | 7.33 |
| dimethylphenyl)hexyl]benzyl}azetidine-3-carboxylic acid | |
| 1-{4-[5-(3-chlorophenyl)-6-(3,4- | 64.54 |
| dimethylphenyl)hexyl]benzyl}azetidine-3-carboxylic acid | |

What is claimed is:

1. A compound represented by Formula I, its individual enantiomers, individual diastereoisomers, individual tautomers or a pharmaceutically acceptable salt thereof 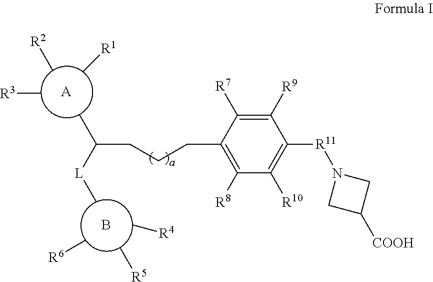
wherein:
A is substituted or unsubstituted C₆₋₁₀ aryl, substituted or unsubstituted heterocycle, substituted or unsubstituted C₃₋₈ cycloalkyl, or substituted or unsubstituted C₃₋₈ cycloalkenyl;
B is substituted or unsubstituted C₆₋₁₀ aryl, substituted or unsubstituted heterocycle, substituted or unsubstituted C₃₋₈ cycloalkyl, or substituted or unsubstituted C₃₋₈ cycloalkenyl;
R¹ is H, halogen, —OR¹², C₁₋₈ alkyl, CN, C(O)R¹³ or NR¹⁴R¹⁵;
R² is H, halogen, —OR¹², C₁₋₈ alkyl, CN, C(O)R¹³ or, NR¹⁴R¹⁵;
R³ is H, halogen, —OR¹², C₁₋₈ alkyl, CN, C(O)R¹³ or NR¹⁴R¹⁵;
R⁴ is H, halogen, —OR¹², C₁₋₈ alkyl, CN, C(O)R¹³ or NR¹⁴R¹⁵;
R⁵ is H, halogen, —OR¹², C₁₋₈ alkyl, CN, C(O)R¹³ or NR¹⁴R¹⁵;
R⁶ is H, halogen, —OR¹², C₁₋₈ alkyl, CN, C(O)R¹³ or NR¹⁴R¹⁵;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;
R¹¹ is O, S or CH₂;
L is CHR¹⁶, O, S or NR¹⁷;
a is 1, 2, 3 or 4;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl;
R¹⁶ is H, OH or C₁₋₈ alkyl; and
R¹⁷ is H or C₁₋₈ alkyl.

2. A compound according to claim 1, wherein:

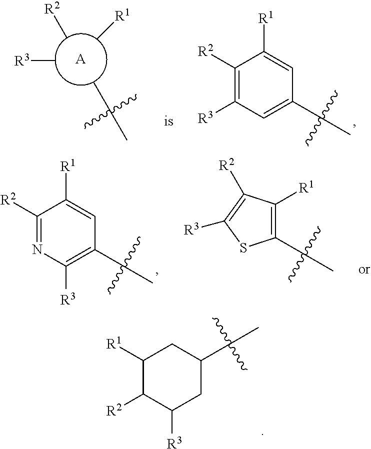

3. A compound according to claim 1, wherein:

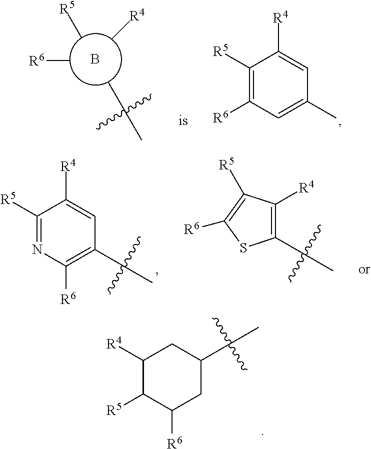

4. A compound according to claim 1, wherein:

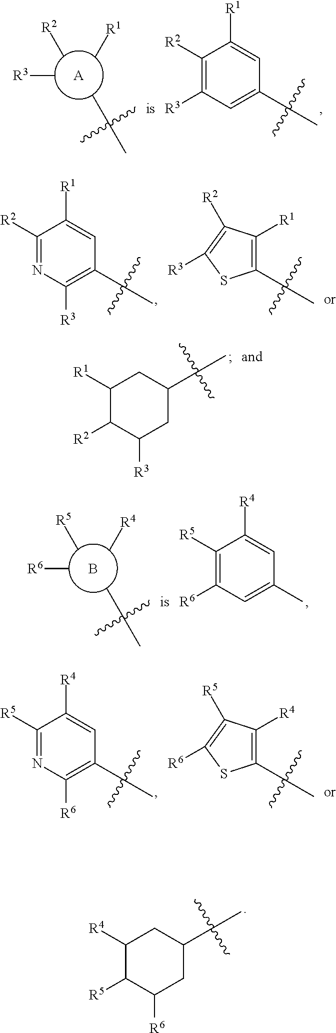

5. A compound according to claim 1, wherein: 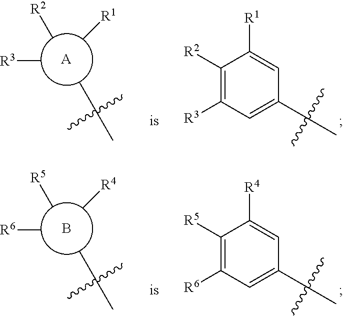
R¹ is H, halogen, —OR¹², C₁₋₈ alkyl, CN, C(O)R¹³ or NR¹⁴R¹⁵;
R² is H, halogen, —OR¹², C₁₋₈ alkyl, CN, C(O)R¹³ or, NR¹⁴R¹⁵;
R³ is H, halogen, —OR¹², C₁₋₈ alkyl, CN, C(O)R¹³ or NR¹⁴R¹⁵;
R⁴ is H, halogen, —OR¹², C₁₋₈ alkyl, CN, C(O)R¹³ or NR¹⁴R¹⁵;
R⁵ is H, halogen, —OR¹², C₁₋₈ alkyl, CN, C(O)R¹³ or NR¹⁴R¹⁵;
R⁶ is H, halogen, —OR¹², C₁₋₈ alkyl, CN, C(O)R¹³ or NR¹⁴R¹⁵;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;
R¹¹ is O, S or CH₂;
L is CHR¹⁶, O, S or NR¹⁷;
a is 1, 2, 3 or 4;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl;
R¹⁶ is H, OH or C₁₋₈ alkyl; and
R¹⁷ is H or C₁₋₈ alkyl.

6. A compound according to claim 1, wherein: 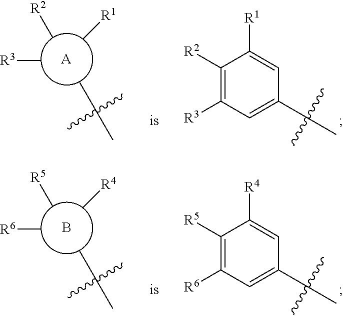
R¹ is H, halogen;
R² is H, halogen;
R³ is H, halogen;
R⁴ is H, C₁₋₈ alkyl;
R⁵ is H, C₁₋₈ alkyl;
R⁶ is H, C₁₋₈ alkyl;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;
R¹¹ is CH₂;
L is CHR¹⁶;
a is 1 or 2;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl; and
R¹⁶ is H, OH or C₁₋₈ alkyl.

7. A compound according to claim 1, wherein: 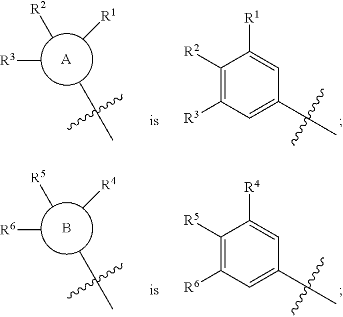
R¹ is H, halogen;
R² is H, halogen;
R³ is H, halogen;
R⁴ is H, C₁₋₈ alkyl;
R⁵ is H, C₁₋₈ alkyl;
R⁶ is H, C₁₋₈ alkyl;
R⁷ is H, halogen, C₁₋₈ alkyl;
R⁸ is H, halogen, C₁₋₈ alkyl;
R⁹ is H, halogen, —C₁₋₈ alkyl;
R¹⁰ is H, halogen, —C₁₋₈ alkyl;
R¹¹ is CH₂;
L is CHR¹⁶;
a is 1 or 2; and
R¹⁶ is H.

8. A compound according to claim 1, wherein: 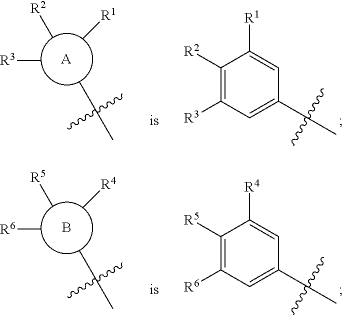
R¹ is halogen;
R² is H;
R³ is H, halogen;
R⁴ is C₁₋₈ alkyl;
R⁵ is C₁₋₈ alkyl;
R⁶ is H;
R⁷ is H;
R⁸ is H;
R⁹ is H;
R¹⁰ is H;
R¹¹ is CH₂;
L is CHR¹⁶;
a is 1 or 2; and
R¹⁶ is H.

9. A compound according to claim 1, wherein: 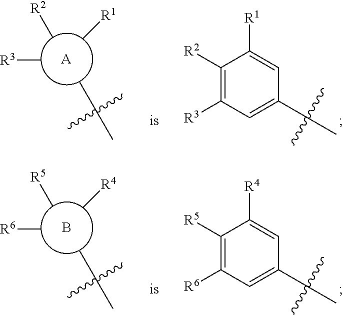
R¹ is Cl or F;
R² is H;
R³ is H or F;
R⁴ is methyl;
R⁵ is methyl;
R⁶ is H;
R⁷ is H;
R⁸ is H;
R⁹ is H;
R¹⁰ is H;
R¹¹ is CH₂;
L is CHR¹⁶;
a is 1 or 2; and
R¹⁶ is H.

10. A compound according to claim 1 which is selected from:
1-{4-[4-(3-chlorophenyl)-5-(3,4-dimethylphenyl)pentyl]benzyl}azetidine-3-carboxylic acid;
1-{4-[5-(3,5-difluorophenyl)-6-(3,4-dimethylphenyl)hexyl]benzyl}azetidine-3-carboxylic acid; and
1-{4-[5-(3-chlorophenyl)-6-(3,4-dimethylphenyl)hexyl]benzyl}azetidine-3-carboxylic acid.

11. A pharmaceutical composition comprising as active ingredient a therapeutically effective amount of a compound according to claim 1 and a pharmaceutically acceptable adjuvant, diluents or carrier.

12. A pharmaceutical composition according to claim 11 wherein the compound is which is selected from:
1-{4-[4-(3-chlorophenyl)-5-(3,4-dimethylphenyl)pentyl]benzyl}azetidine-3-carboxylic acid;
1-{4-[5-(3,5-difluorophenyl)-6-(3,4-dimethylphenyl)hexyl]benzyl}azetidine-3-carboxylic acid; and
1-{4-[5-(3-chlorophenyl)-6-(3,4-dimethylphenyl)hexyl]benzyl}azetidine-3-carboxylic acid.

13. A method of treating a disorder associated with sphingosine-1-phosphate receptor modulation, which comprises administering to a mammal in need thereof, a pharmaceutical composition comprising a therapeutically effective amount of at least one compound of Formula I 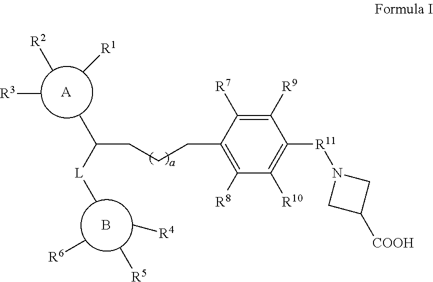
wherein:
A is substituted or unsubstituted C₆₋₁₀ aryl, substituted or unsubstituted heterocycle, substituted or unsubstituted C₃₋₈ cycloalkyl, or substituted or unsubstituted C₃₋₈ cycloalkenyl;
B is substituted or unsubstituted C₆₋₁₀ aryl, substituted or unsubstituted heterocycle, substituted or unsubstituted C₃₋₈ cycloalkyl, or substituted or unsubstituted C₃₋₈ cycloalkenyl;
R¹ is H, halogen, —OR¹², C₁₋₈ alkyl, CN, C(O)R¹³ or NR¹⁴R¹⁵;
R² is H, halogen, —OR¹², C₁₋₈ alkyl, CN, C(O)R¹³ or, NR¹⁴R¹⁵;
R³ is H, halogen, —OR¹², C₁₋₈ alkyl, CN, C(O)R¹³ or NR¹⁴R¹⁵;
R⁴ is H, halogen, —OR¹², C₁₋₈ alkyl, CN, C(O)R¹³ or NR¹⁴R¹⁵;
R⁵ is H, halogen, —OR¹², C₁₋₈ alkyl, CN, C(O)R¹³ or NR¹⁴R¹⁵;
R⁶ is H, halogen, —OR¹², C₁₋₈ alkyl, CN, C(O)R¹³ or NR¹⁴R¹⁵;
R⁷ is H, halogen, —OR¹², C₁₋₈ alkyl, CN, C(O)R¹³ or NR¹⁴R¹⁵;
R⁸ is H, halogen, —OR¹², C₁₋₈ alkyl, CN, C(O)R¹³ or NR¹⁴R¹⁵;
R⁹ is H, halogen, —OR¹², C₁₋₈ alkyl, CN, C(O)R¹³ or NR¹⁴R¹⁵;
R¹⁰ is H, halogen, —OR¹², C₁₋₈ alkyl, CN, C(O)R¹³ or NR¹⁴R¹⁵;
R¹¹ is O, S or CH₂;
L is CHR¹⁶, O, S or NR¹⁷;
a is 1, 2, 3 or 4;
R¹² is H or C₁₋₈ alkyl;
R¹³ is H, —OC₁₋₈ alkyl, OH, NR¹⁴R¹⁵ or C₁₋₈ alkyl;
R¹⁴ is H or C₁₋₈ alkyl;
R¹⁵ is H or C₁₋₈ alkyl;
R¹⁶ is H, OH or C₁₋₈ alkyl; and
R¹⁷ is H or C₁₋₈ alkyl.

14. A method of claim 13, wherein the pharmaceutical composition is administered to the mammal to treat ocular diseases, wet and dry age-related macular degeneration, diabetic retinopathy, retinopathy of prematurity, retinal edema, geographic atrophy, glaucomatous optic neuropathy, chorioretinopathy, hypertensive retinopathy, ocular ischemic syndrome, prevention of inflammation-induced fibrosis in the back of the eye, various ocular inflammatory diseases including uveitis, scleritis, keratitis, and retinal vasculitis; or systemic vascular barrier related diseases, various inflammatory diseases, including acute lung injury, its prevention, sepsis, tumor metastasis, atherosclerosis, pulmonary edemas, and ventilation-induced lung injury; or autoimmune diseases and immunosuppression, rheumatoid arthritis, Crohn's disease, Graves' disease, inflammatory bowel disease, multiple sclerosis, Myasthenia gravis, Psoriasis, ulcerative colitis, autoimmune uveitis, renal ischemia perfusion injury, contact hypersensitivity, atopic dermatitis, and organ transplantation; or allergies and other inflammatory diseases, urticaria, bronchial asthma, and other airway inflammations including pulmonary emphysema and chronic obstructive pulmonary diseases; or cardiac protection, ischemia reperfusion injury and atherosclerosis; or wound healing such as but not limited to: scar-free healing of wounds from cosmetic skin surgery, ocular surgery, GI surgery, general surgery, oral injuries, various mechanical, heat and burn injuries, prevention and treatment of photoaging and skin ageing, and prevention of radiation-induced injuries; or bone formation, treatment of osteoporosis and various bone fractures including hip and ankles; or anti-nociceptive activity, visceral pain, pain associated with diabetic neuropathy, rheumatoid arthritis, chronic knee and joint pain, tendonitis, osteoarthritis, neuropathic pains; or central nervous system neuronal activity in Alzheimer's disease, age-related neuronal injuries; or organ transplant such as renal, corneal, cardiac or adipose tissue transplant.

15. The method of claim 13 wherein the mammal is a human.
